(19) United States
(12) Patent Application Publication
(10) Pub. No.: US 2014/0148709 A1
(43) Pub. Date: May 29, 2014

(54) SYSTEM AND METHOD FOR INTEGRATING HEART RATE MEASUREMENT AND IDENTITY RECOGNITION

(71) Applicants: Ren-Hau Gu, Hsin-Chu (TW); Ming-Tsan Kao, Hsin-Chu (TW); Sen-Huang Huang, Hsin-Chu (TW)

(72) Inventors: Ren-Hau Gu, Hsin-Chu (TW); Ming-Tsan Kao, Hsin-Chu (TW); Sen-Huang Huang, Hsin-Chu (TW)

(73) Assignee: PixArt Imaging Incorporation

(21) Appl. No.: 13/684,412

(22) Filed: Nov. 23, 2012

(51) Int. Cl. A61B 5/024; A61B 5/117; A61B 5/00

(52) U.S. Cl. CPC A61B 5/024; A61B 5/0077; A61B 5/117; A61B 5/7278; A61B 5/1176; A61B 5/1172
USPC 600/479

(57) ABSTRACT

A system for integrating heart rate measurement and identity recognition is provided. The system includes a storage unit, an image sensor, an identity recognition unit and a frequency extractor. The storage unit stores a user information database. The image sensor captures a plurality of skin images of a user during a time period. The identity recognition unit receives a physiological feature from the user and recognizes the user by comparing the physiological feature with the user information database in the storage unit. The frequency extractor obtains a frequency signal by extracting a motion signal from the skin images according to the brightness change of the different color lights in the skin images during a time period. The frequency signal represents a frequency in a specific frequency interval, and it represents heart rate information of the user. A method for use in the above-mentioned system is also disclosed.

### CROSS REFERENCE

The present invention claims priority to TW 100143849, filed on Nov. 30, 2011.

### BACKGROUND OF THE INVENTION

1. Field of Invention

The present invention relates to a system and method, especially to a system and method for integrating heart rate measurement and identity recognition.

2. Description of Related Art

Cardiovascular disease is a highly risky threat to human beings. Nowadays, life is full of busy schedules, and heart easily overloads when everything is in a hurry. However, people are used to ignoring the importance of rest and care for this important organ. Heart works silently until it is no longer capable of keeping it, then the cardiovascular disease blows up. The heart at this exhausted stage can no longer recover back to the original healthy status, and people with cardiovascular disease can only live cautiously to avoid worst situation. Even so, heart attack often still occurs and is quite unavoidable. If heart attack happens without proper treatment in time, the result could be a tragedy.

Generally speaking, in order to accurately monitor and record people' s heart rate, usually it requires expensive measuring equipments such as pulse oximeter. These equipments are costly, and require manual operations to record user information and measured data. This is very inconvenient to a user.

### SUMMARY OF THE INVENTION

The present invention provides a system for integrating heart rate measurement and identity recognition, which is advantageous in easy operation, accurate detection of heart rate, and low cost.

The present invention also provides a method for integrating heart rate measurement and identity recognition, for use in the foregoing system to provide the same benefits.

The above and other objectives and advantages of the present invention can be further understood from the disclosed technical features in the invention.

To achieve the above or other objectives, one preferable embodiment of the present invention provides a system for integrating heart rate measurement and identity recognition. The system comprises a storage unit, an image sensor, an identity recognition unit and a frequency extractor. The storage unit includes a user information database. The image sensor captures a plurality of skin images of a user. The identity recognition unit receives a physiological feature from the user and compares it with data in the user information database in the storage unit, to recognize the user. The frequency extractor obtains a frequency signal by extracting a motion signal from the skin images according to the brightness changes of the different color lights in the skin images during a period of time. The frequency signal is a frequency in a specific frequency interval and it represents heart rate information of the user.

In a preferable embodiment of the present invention, the heart rate information is stored in the storage unit to update the user information database.

In a preferable embodiment of the present invention, the skin images include a plurality of facial images, a plurality of fingerprint images, or a plurality of images of a body part with a vein.

In a preferable embodiment of the present invention, the physiological feature includes a facial feature, a fingerprint feature, a vein pattern feature, or a phonetic feature.

In a preferable embodiment of the present invention, the identity recognition unit recognizes the user by comparing the skin images from the image sensor with data in the user information database in the storage unit, wherein the skin images are a physiological feature recognizable by the identity recognition unit.

In a preferable embodiment of the present invention, the physiological feature is a facial feature, and the skin images are facial images of the user.

In a preferable embodiment of the present invention, the system further comprises a face detecting and tracking unit, which checks whether the skin images belong to a part of the user's face according to a face detecting and tracking algorithm.

In one embodiment of the present invention, the identity recognition unit and the frequency extractor are integrated in one processor. In another embodiment, they are performed by different processors.

In a preferable embodiment of the present invention, the specific frequency interval corresponds to a heart rate frequency of the user, which is between 0 to 5 Hz.

In a preferable embodiment of the present invention, the frequency extractor extracts the motion signal according to ICA (Independent Component Analysis) algorithm.

Another objective of the present invention is to provide a method for integrating heart rate measurement and identity recognition. The method comprises: capturing a plurality of skin images of a user; and obtaining a frequency signal by extracting a motion signal from the skin images according to brightness changes of different color lights in the skin images during a period of time. The frequency signal is a frequency in a specific frequency interval and it represents heart rate information of a user.

In a preferable embodiment of the present invention, the foregoing method further comprises: recognizing the user by comparing a physiological feature of the user with data in a user information database.

In a preferable embodiment of the present invention, the foregoing method further comprises: storing the heart rate information into the user information database to update the user information database.

In a preferable embodiment of the present invention, a skin image of the user is one of the physiological features, and the method further comprising: recognizing the user by comparing one or more skin images of the user with data in the user information database.

In a preferable embodiment of the present invention, the foregoing method further comprises: checking whether the skin images belong to a part of the user's face according to a face detecting and tracking algorithm.

In a preferable embodiment of the present invention, the step of obtaining the frequency signal by extracting the motion signal from the skin images is performed by Independent Component Analysis.

According to the above, the present invention is capable of recognizing a user by identity recognition unit, and further obtaining heart rate information of the user. An image sensor captures plural skin images, and a frequency extractor extracts a motion signal from the plural skin images according to brightness changes of different color lights in the skin images during a period of time, to obtain a frequency signal. The frequency signal represents heart rate information of the user. In another embodiment, the plural skin images captured by the image sensor is sent to an identity recognition unit for user identity recognition on the one hand, and also sent to a frequency extractor for frequency extraction on the other hand. The frequency extractor extracts a motion signal from of the plural skin images according to brightness changes of different color lights in the skin images. The frequency signal represents heart rate information of the user; the heart rate information is stored in a user information database. In summary, the present invention provides a system having the benefits of easy operation, low cost, and precise heart rate detection. The present invention also provides a method for integrating heart rate measurement and identity recognition with such features and benefits.

The objectives, technical details, features, and effects of the present invention will be better understood with regard to the detailed description of the embodiments below, with reference to the drawings.

### BRIEF DESCRIPTION OF THE DRAWINGS

FIG. 1 shows an embodiment of a system for integrating heart rate measurement and identity recognition according to the present invention.

FIG. 2 shows that a user's multiple skin images are sensed by the image sensor during a period of time in FIG. 1.

FIG. 3A˜FIG. 3C respectively show statistical data of red, green, and blue compositions of the skin images during a period of time T1.

FIG. 4A˜FIG. 4C respectively show the Independent Component Analysis results according to the data shown in FIG. 3A˜FIG. 3C.

FIG. 5A˜FIG. 5C show the frequency-domain transformation obtained from the data of FIG. 4A˜FIG. 4C.

FIG. 6 shows the flowchart of a method for integrating heart rate measurement and identity recognition according to an embodiment of the present invention.

FIG. 7 shows a system for integrating heart rate measurement and identity recognition according to an embodiment of the present invention.

FIG. 8 shows the flowchart of a method for integrating heart rate measurement and identity recognition according to another embodiment the present invention.

### DESCRIPTION OF THE PREFERRED EMBODIMENTS

The drawings as referred to throughout the description of the present invention are for illustration only, but not drawn according to actual scale. The orientation wordings in embodiment descriptions such as: top, bottom, left, and right are for reference only, but not for limiting the scope of the present invention.

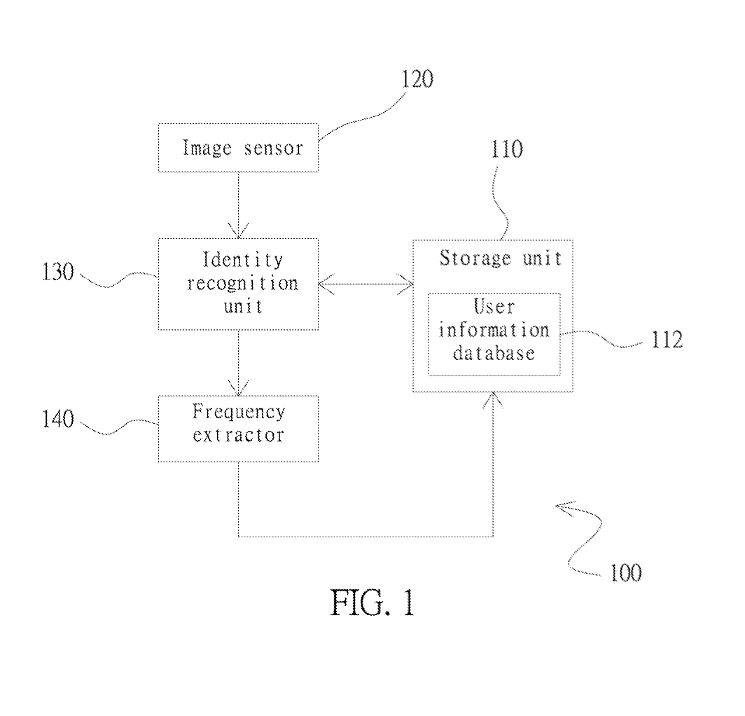

Referring to FIG. 1, a system for integrating heart rate measurement and identity recognition according to a first embodiment of the present invention is illustrated. The disclosed system 100 comprises a storage unit 110, an image sensor 120, an identity recognition unit 130 and a frequency extractor 140. The storage unit 110 includes a user information database 112. In this embodiment, the storage unit 110 can be a memory, floppy disk, hard disk CD, flash drive, tape, network accessible database, or any other storage media with storage function. The user information database 112 stores user data, such as: name, address, telephone number, personal medical history information, or heart rate history records. In this embodiment, the user information database 112 can be in different forms corresponding to the types of the storage unit 110. For example, if the storage unit 110 is a remote data server, then the user information database 112 can for example be a cloud database. In this case the system 100 receives or stores the user information in the user information database 112 by wired or wireless communication with the storage unit 110. In another case, the storage unit 110 can be an electronic device integrated within the system 100. The above are only examples and they can be implemented in any way according to the user's requirements.

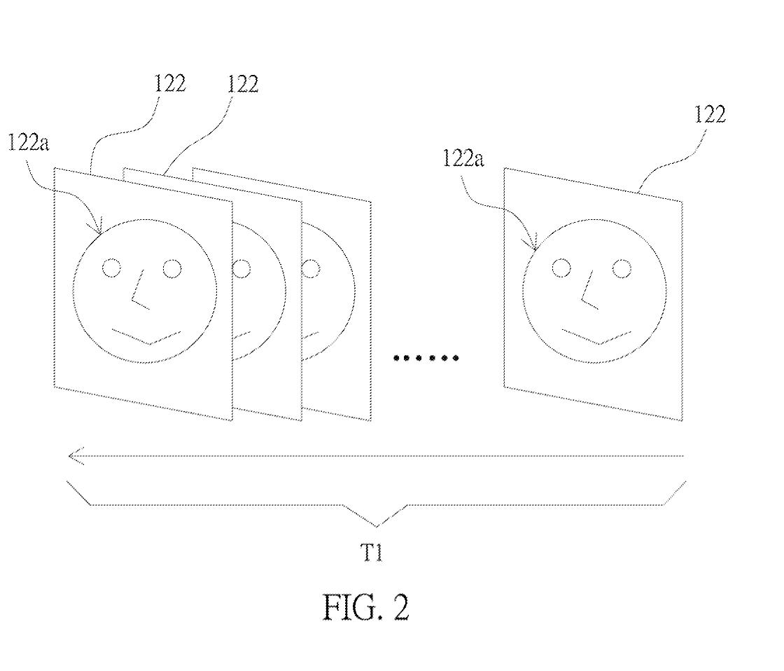

In the system 100, the image sensor 120 provides a function of capturing a plurality of skin images 122 of a user 122a during a period of time T1. As shown in FIG. 2, the skin images 122 are for example facial images, i.e., images of a face, but the present invention is not limited to this example. In other embodiments, the skin images 122 can be fingerprint images or images of another body part with a vein. And, the image sensor 120 can be a CCD (charge coupled device) or a CMOS image sensor (complementary metal oxide semiconductor image sensor). As a further example, the image sensor 120 can be a Webcam or any other device capable of capturing images.

The identity recognition unit 130 provides a function of recognizing a user 122a by receiving a physiological feature from the user 122a, and comparing it with the user information database 112 in the storage unit 110. As specific examples, the physiological feature can be a facial feature, a fingerprint feature, a vein pattern feature, or a phonetic feature, etc., and the identity recognition unit 130 recognizes the user's identity according to one or more of these features. One or more of these “physiological features” are stored in the user information database 112 within the storage unit 110, so that the identity recognition unit 130 can compare a captured/sensed physiological feature with the stored physiological feature.

For example, after the identity recognition unit 130 receives the user's feature such as face, fingerprint, vein pattern, or phonetic feature, the identity recognition unit 130 can compare it with the data stored in the user information database 112, to determine/recognize the user's identity. In this embodiment, optionally, if the skin image 122 (for example: a facial image or fingerprint image) sensed by the image sensor 120 is a physiological feature recognizable by identity recognition unit 130, the identity recognition unit 130 will proceed to recognize the user's identity by comparing the skin image 122 captured by the image sensor 120 with the user information database 112 in the storage unit 110. That is, when the physiological feature is a facial or fingerprint feature, and the skin image 122 happens to be the user's facial image or fingerprint image, then the identity recognition unit 130 will directly compare the skin image 122 from the image sensor 120 with the user information database 112 in the storage unit 110 for identity recognition.

In this embodiment, after the image sensor 120 captures the skin images 122 of the user 122a, the frequency extractor 140 extracts a motion signal from the skin images 122 according to brightness changes of different color lights in the skin images 122 during a period of time T1, to obtain a frequency signal S1. The frequency signal S1 is a frequency in a specific frequency interval F1 and it can represent heart rate information of a user. In one embodiment, the frequency extractor 140 extracts the motion signal to obtain the above-mentioned frequency signal by ICA (Independent Component Analysis).

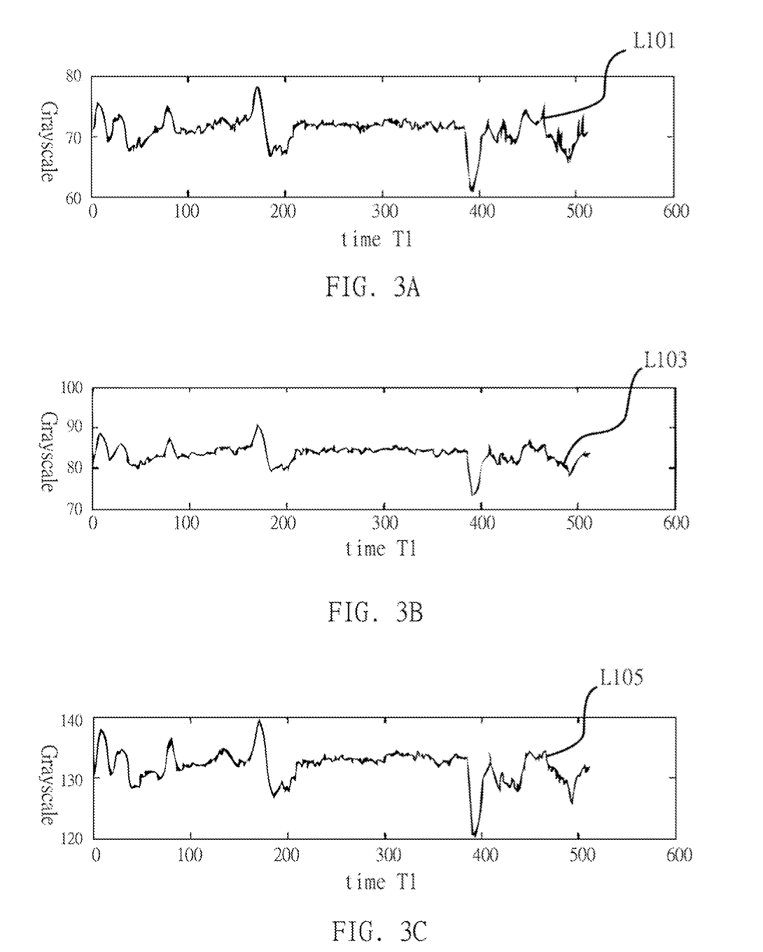

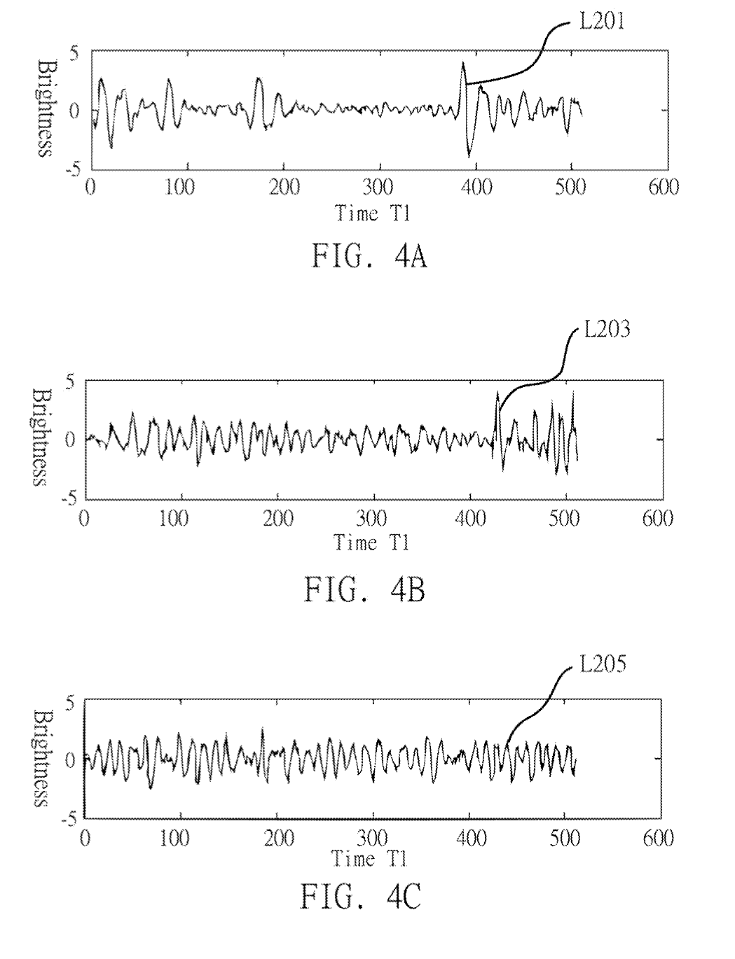

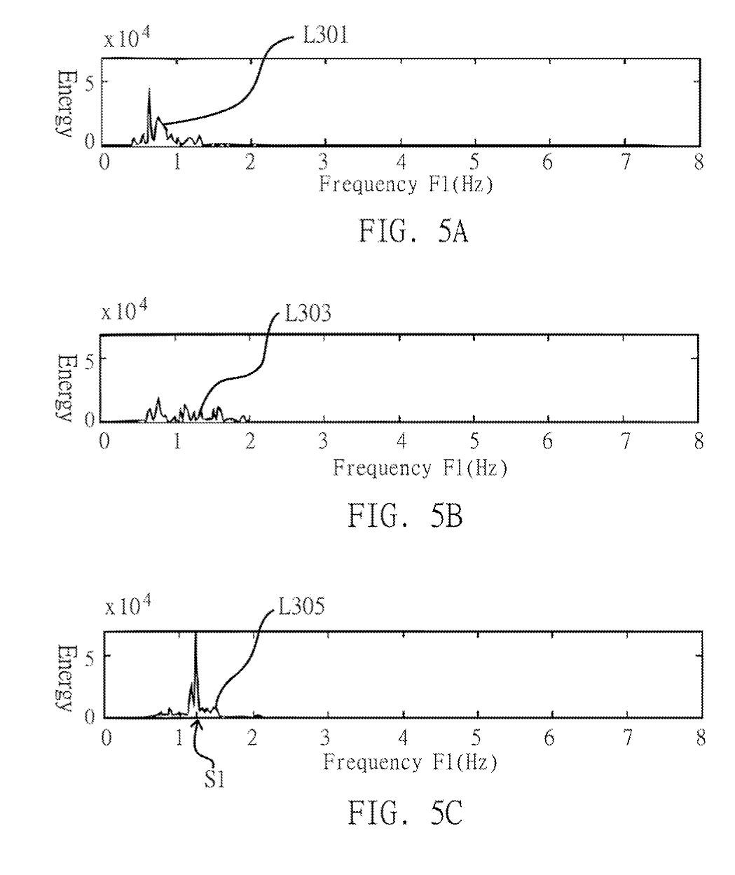

For illustrating purpose, FIGS. 3A˜3C, FIGS. 4A˜4C, and FIGS. 5A˜5C show an example of the operating process of the frequency extractor 140 for obtaining the frequency signal of heart rate information. FIGS. 3A˜3C respectively illustrate statistical data of the red, green and blue compositions of the skin images in the period of time T1. FIGS. 4A˜4C show Independent Component Analysis results according to the data shown in FIGS. 3A˜3C. FIGS. 5A˜5C show the frequency-domain transformation obtained from the data of FIGS. 4A˜4C.

Refer to FIGS. 3A˜3C, the frequency extractor 140 obtains red composition statistical data, green composition statistical data and blue composition statistical data after processing the captured skin images during the period of time T1. As an example for how the red composition statistical data, the green composition statistical data and the blue composition statistical data are obtained, the frequency extractor 140 extracts the red composition, green composition and blue composition from the skin images 122, and then obtains plural red composition images, plural green composition images, and plural blue composition images.

Next, the frequency extractor 140 can obtain red composition data by processing the red composition images. After processing plural red composition images, plural red composition data are obtained, as shown by the curve L101 in FIG. 3A. In each of FIGS. 3A˜3C, the horizontal axis is the timeline, and the vertical axis is the grayscale index. In a similar way, the frequency extractor 140 processes each green composition image to obtain a corresponding green composition data. When all of the green composition images are processed, plural green composition data are obtained, as shown by the curve L103 in FIG. 3B. Likewise, the frequency extractor 140 processes each blue composition image to obtain a corresponding blue composition data. When all of the blue composition images are processed, plural blue composition data are obtained, as shown by the curve L105 in FIG. 3C.

For example, the red composition data can be average brightness, maximum brightness, minimum brightness, or other statistical data corresponding to the red composition image; the green composition data can be average brightness, maximum brightness, minimum brightness, or other statistical data corresponding to the green component image; the blue composition data can be average brightness, maximum brightness, minimum brightness, or other statistical data corresponding to the blue composition image.

And, the frequency extractor 140 respectively processes the red composition data, green composition data and the blue composition data shown in FIGS. 3A˜3C by Independent Component Analysis, to obtain a first composition data, a second composition data, and a third composition data as respectively shown by curves L201, L203, and L205 in. FIGS. 4A˜4C. Independent Component Analysis is a known algorithm: when three sets of data are inputted, Independent Component Analysis will produce three sets of output data respectively. The words “first”, “second” and “third” in the terms “first composition data”, “second composition data” and “third composition data” are for distinguishing these data from one another as different composition data, but do not imply any sequential order.

In one embodiment of the present invention, Independent Component Analysis is used to extract the human heart rate signal and other signals (such as the aforementioned motion signal) in the red composition data, the green composition data, and the blue composition data. In other words, one of the first composition data, the second composition data, and the third composition data can be regarded as the human heart rate signal. Human heart rate signal shows a special characteristic in frequency domain, that is, it has a higher energy in a specific frequency interval. In this embodiment, a predetermined frequency interval is set in correspondence to this frequency interval. Generally, most human heart rates have a low frequency, and therefore the predetermined frequency interval can be set to a low frequency range.

Next, frequency extractor 140 converts the first composition data, the second composition data and the third composition data shown in FIGS. 4A˜4C into frequency domain, to obtain first frequency-domain information, second frequency-domain information, and third frequency-domain information, which are respectively shown in FIGS. 5A˜5C as curves L301, L303, L305. The horizontal axis in each of FIGS. 5A˜5C represents frequency index, and the vertical axis represents energy index. As an example, the conversion can be done by FFT (Fast Fourier Transform).

Next, the frequency extractor 140 compares the energy indices of the first, second and third frequency-domain data in a specific frequency interval, to obtain the user's heart rate information, wherein the frequency signal S1 corresponding to the highest energy index is the user's heart rate information. As shown by FIGS. 5A˜5C, FIG. 5C has the highest energy index, and the frequency signal S1 corresponding to this energy index is 1.23 Hz. Because 1 Hz is equivalent to 60 heartbeat/min, the user's heart rate is 73.82 heartbeats per minute. In this embodiment, the specific frequency interval F1 corresponds to the range of the user's heart rate, and the specific frequency interval F1 is between 0˜5 Hz.

Further, the foregoing heart rate information of the user during the period of time T1 can be selectively stored in the storage unit 110 to update the user information in the user information database 112.

The operation of the foregoing identity recognition unit 130 and the frequency extractor 140 can belong to the same processor, or different processors. In addition, the above system 100 can also include a face detecting and tracking unit (not shown in figures), which can check whether a skin image 122 belongs to a part of the user's facial image by a face detecting and tracking algorithm.

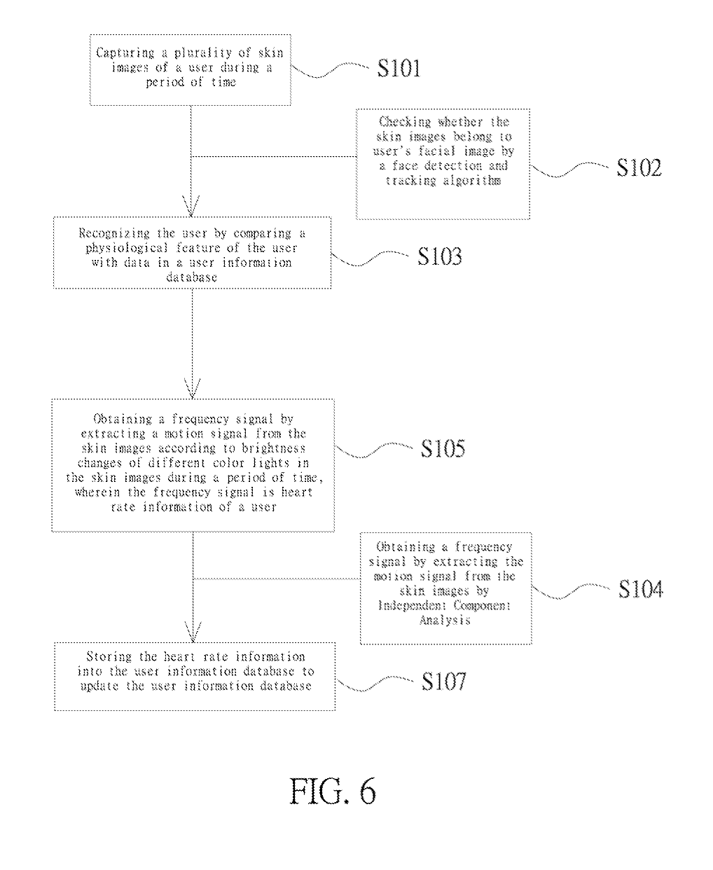

Based on the above, the present invention also provides a method for integrating heart rate measurement and identity recognition, as shown in FIG. 6. Referring to the step S101 shown in FIG. 1 and FIG. 6, first, capture a plurality of skin images 122 of the user during a period of time T1. The skin images 122 can be captured for example by an image sensor 120 as mentioned in the above. Optionally, the method can check whether the skin images 122 belong to the user's face by a face detecting and tracking algorithm, as shown by the step S102 in FIG. 6.

Next, the user is recognized by receiving a physiological feature of the user and comparing it with the user information database 112 which is described with reference to FIG. 1, as shown by the step S103 in FIG. 6. The “physiological feature” can be facial feature, fingerprint feature, vein pattern feature, or phonetic feature, etc. When the skin image 122 happens to be a recognizable physiological feature of the user, such as a facial image, the skin image 122 can be directly compared with the user information database 112 in the storage unit 110 to recognize the user.

And, a frequency signal S1 is obtained by extracting a motion signal from the skin images according to brightness changes of different color lights in the skin images during a period of time, wherein the frequency signal S1 is located in a specific frequency interval F1 and it represents the user's heart rate information, as shown in FIG. 1 and the step S105 in FIG. 6. Specifically, the frequency signal S1 can be obtained by the aforementioned ICA to extract the motion signal from these skin images 122, as shown in step S104.

Then, the heart rate information of the user in the period of time T1 is stored in the user information database 112 to update the user information database 112, as shown in FIG. 1 and the step S107 of FIG. 6. Thus, the steps for integrating integrated heart rate measurement and identity recognition are substantially completed.

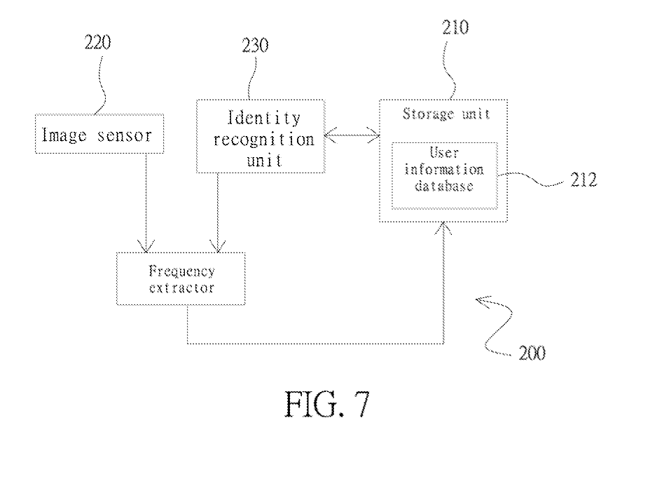

FIG. 7 shows another embodiment of the system for integrating heart rate measurement and identity recognition according to the present invention is shown. Referring to FIG. 1 in conjunction with FIG. 7, the system 200 is similar to the aforementioned system 100 in concept, but they are different in that: the skin image 122 captured by the image sensor 120 in the system 100 can also be used for user identity recognition by the identity recognition unit 130 if the skin image 122 is a recognizable physiological feature; however, in system 200, an image sensor 220 and an identity recognition unit 230 work separately. For example, the identity recognition unit 230 identifies a user by voice recognition, and the skin images of the user's body parts are captured by the image sensor 220 and provided to a frequency extractor 240 for analysis to obtain the user's heart rate information. That is, the identity recognition unit 230 may recognize the user's identity not by the images captured by image sensor 220. The heart rate information can be sent to the user information database 212 to update or record it. It should be noted that the priority between the operations of the image sensor 220 and the identity recognition unit 230 can be arranged according to requirements. In this embodiment, the user's identity is recognized by the identity recognition unit 230 first, by voice, fingerprint, or other means, and then the image sensor 220 captures the user's skin images of a body part. In another embodiment, it can be arranged so that the user's skin images of a body part is first captured, and then the user' s identity is recognized by the identity recognition unit 230 by voice, fingerprints, or other means.

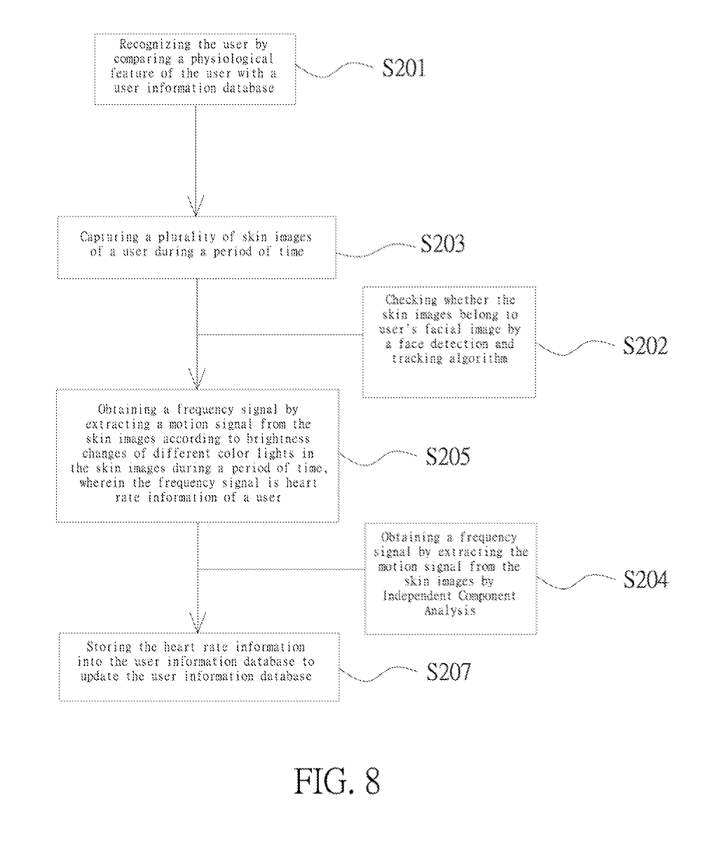

According to the above, another embodiment of a method for integrating heat rate measurement and identity recognition according to the present invention is provided, as shown in FIG. 8. Referring to FIG. 6 and FIG. 8, this method embodiment of the is similar to the aforementioned method embodiment in concept, but they are different in that: in this method embodiment, the recognition of the user's identity by the physiological feature, and the analysis and storage of the user's heart rate information by the user's skin images are performed separately, as shown from step S201 through step S207. In this embodiment, the captured skin images are not be used to recognize the user, which is the difference from the previous method.

In summary, the system and method for integrating heart rate measurement and identity recognition according to the present invention have at least the following features: the user's identity can be identified by an identity recognition unit; a plurality of skin images of the user can be captured by a image sensor, and a frequency signal is obtained by a frequency extractor by extracting a motion signal from the skin images according to brightness changes of different color lights in the skin images, wherein the frequency signal represents heart rate information of the user. Then, the heart rate information of the user can be stored in a user information database. Thus, the user can be automatically recognized and the measured data can be automatically recorded. As such, the system has the benefits of easy operation and accurate heart rate detection, at low cost.

In addition, the skin images captured by the image sensor also can be used for user identity recognition; in this case the skin images are on the one hand used for user identity recognition, on the other hand used by the frequency extractor to obtain a frequency signal, wherein the frequency extractor extracts a motion signal from the skin images according to brightness changes of different color lights in the skin images, and obtains the frequency signal from the motion signal, wherein the frequency signal represents heart rate information. The heart rate information of the user can be stored in the user information database, so that the user can be automatically recognized and the measured data can be automatically recorded. Besides, the present invention also provides a method for integrating heart rate measurement and identity recognition with the foregoing features and benefits.

The present invention has been described in considerable detail with reference to certain preferred embodiments thereof. It should be understood that the description is for illustrative purpose, not for limiting the scope of the present invention. Those skilled in this art can readily conceive variations and modifications within the spirit of the present invention, which should belong to the scope of the present invention. One embodiment or one claim of the present invention does not have to achieve all the objectives or advantages or include all the features of the present invention. The title and the abstract are provided for assisting searches and should not be read as limitations to the present invention.

What is claimed is:

1. A system for integrating heart rate measurement and identity recognition, comprising:
an image sensor, for capturing a plurality of skin images of a user; and
a frequency extractor, for obtaining a frequency signal by extracting a motion signal from the skin images according to brightness changes of different color lights in the skin images during a period of time.

2. A system of claim 1, wherein the frequency signal is in a specific frequency interval, and represents heart rate information of a user.

3. A system of claim 2, further comprising:
a storage unit, for storing a user information database; and
an identity recognition unit, for receiving a physiological feature of the user and comparing the physiological feature with data in the user information database to recognize the user.

4. A system of claim 3, wherein the heart rate information is stored in the storage unit to update the user information database.

5. A system of claim 1, wherein the skin images comprise a plurality of facial images, a plurality of fingerprint image, or a plurality of images of a body part with a vein.

6. A system of claim 3, wherein the physiological feature comprises a facial feature, a fingerprint feature, a vein pattern feature, or a phonetic feature.

7. A system of claim 3, wherein the identity recognition unit recognizes the user by comparing the skin images from the image sensor with data in the user information database in the storage unit.

8. A system of claim 7, wherein the user information database stores facial features, and the skin images are facial images of the user.

9. A system of claim 8, further comprising a face detecting and tracking unit, which checks whether the skin images belong to a part of the user's face according to a face detecting and tracking algorithm.

10. A system of claim 3, wherein the identity recognition unit and the frequency extractor are integrated into one processor or separately processed by different processors.

11. A system of claim 2, wherein the specific frequency interval corresponds to a heat rate frequency of the user, which is between 0 to 5 Hz.

12. A system of claim 1, wherein the frequency extractor extracts the motion signal by Independent Component Analysis.

13. A method for integrating heart rate measurement and identity recognition, comprising:
capturing a plurality of skin images of a user; and
obtaining a frequency signal by extracting a motion signal from the skin images according to brightness changes of different color lights in the skin images during a period of time.

14. A method of claim 13, wherein the frequency signal is in a specific frequency interval and represents heart rate information of a user.

15. A method of claim 14, further comprising:
storing the heart rate information into a user information database to update the user information database.

16. A method of claim 13, further comprising:
recognizing the user by comparing a physiological feature of the user with data in a user information database.

17. A method of claim 16, wherein one or more of the skin images comprise the physiological feature of the user, and the method further comprising:
recognizing the user by comparing the one or more skin images of the user with data in the user information database.

18. A method of claim 13, further comprising:
checking whether the skin images belong to a part of the user's face according to a face detecting and tracking algorithm.

19. A method of claim 13, wherein the step of obtaining the frequency signal by extracting the motion signal from the skin images is performed by Independent Component Analysis.
